(19) United States
(12) Patent Application Publication
(10) Pub. No.: US 2009/0191163 A1
(43) Pub. Date: Jul. 30, 2009

(54) Primed tissue for tissue engineering and methods of priming tissue

(76) Inventor: Vincent Falanga, Boston, MA (US)

Correspondence Address: Ballard Spahr Andrews & Ingersoll, LLP, SUITE 1000, 999 PEACHTREE STREET, ATLANTA, GA, 30309-3915, US

(21) Appl. No.: 12/321,819

(22) Filed: Jan. 26, 2009

Related U.S. Application Data
(60) Provisional application No. 61/062,969, filed on Jan. 30, 2008.

(51) Int. Cl. A61K 45/00; C12N 5/06; C12N 5/08; A61P 17/02; A61B 19/02

(52) U.S. Cl. 424/93.7; 435/325; 435/371; 206/438

(57) ABSTRACT

Provided is a composition comprising a primed engineered tissue construct, methods of making the composition, methods of using the composition in dermatologic surgery, and a kit for supplying surgical tissue graft components.

### CROSS REFERENCE TO RELATED APPLICATION

This application claims the benefit of U.S. Provisional Application No. 61/062,969, filed Jan. 30, 2008, which is herein incorporated by reference in its entirety.

### BACKGROUND OF THE INVENTION

1. Field of the Invention

This invention relates generally to the field of dermatologic surgery. Specifically, the invention relates to novel compositions and methods of use thereof for treating acute and chronic wounds.

2. Background Art

In recent years, considerable success in the treatment of non-healing chronic wounds has been achieved with the use of cell-based therapies.¹⁻⁵ In a pivotal trial, a dermal fibroblast living construct led to faster healing of diabetic foot ulcers.^{6, 7} In other controlled trials, it has been shown that a living bi-layered skin construct (BSC)⁸, consisting of allogeneic keratinocytes and fibroblasts from neonatal foreskin, accelerates the healing of both venous and diabetic ulcers.^{9, 10} However, in spite of these advances, it was found that up to 50% of long standing venous ulcers are unresponsive to a BSC.¹¹ The causes of this therapeutic unresponsiveness are unknown, but there is evidence that the construct breaks down in the wound bed before it is fully active and able to stimulate healing.¹² At present, BSC and other living constructs are used within a few minutes after their removal from their transport media. However, it is likely that subjects are not receiving the full benefit of these sophisticated living constructs, whose stimulatory programs for accelerating healing may take time to unfold.

Several bioengineered skin products or skin equivalents have become available for the treatment of acute and chronic wounds, as well as for burns. Since the initial use of more simple keratinocyte sheets,¹³⁻¹⁶ several more complex constructs have been developed and tested in human wounds. The skin equivalents used to treat wounds have generally contained living cells, such as fibroblasts or keratinocytes or both,¹⁷⁻²⁰ while others are made of acellular materials or extracts of living cells.^{21,22} Some allogeneic constructs consisting of living cells derived from neonatal foreskin have been shown to accelerate the healing of chronic wounds in randomized controlled trials, and are commercially available.^{7, 9, 10, 23, 24} The results of these trials, which have led to the FDA approval of two such constructs, have shown an efficacy of between 15% and 20% over control in patients treated with conventional therapy alone.

What is needed in the art is a composition which increases effectiveness in healing chronic wounds. The present invention provides a composition comprising an engineered tissue construct that is primed for a prolonged period of time in vitro before its application to a non-healing wound in a subject. The priming renders the tissue construct more effective at the outset, before wound exudates, bacterial products, matrix metalloproteinases (MMPs), and other complex pathophysiological abnormalities adversely affect its in vivo performance.^{12, 13}

### SUMMARY OF THE INVENTION

In accordance with the purposes of this invention, as embodied and broadly described herein, this invention, in one aspect, relates to a composition comprising a primed engineered tissue construct, wherein the tissue construct is primed by contact with a tissue culture medium in vitro.

In another aspect, the invention relates to a method of priming an engineered tissue construct, comprising contacting the tissue construct with a tissue culture medium, whereby contacting the tissue construct with the tissue culture medium primes the tissue construct.

In yet another aspect, the invention relates to a composition comprising a primed engineered tissue construct, prepared by a method comprising contacting the tissue construct with a tissue culture medium.

In another aspect, the invention relates to a method for healing a wound in a subject, comprising applying to the wound in the subject a therapeutically effective amount of a primed engineered tissue construct, whereby applying the tissue construct to the wound heals the wound in the subject.

In yet another aspect, the invention relates to a kit for supplying surgical tissue graft components, the kit comprising a first compartment comprising an engineered tissue construct and a second compartment comprising a predetermined quantity of a tissue culture medium, wherein the predetermined quantity is sufficient to prime the tissue construct when the tissue construct is in contact with the tissue culture medium.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows a bi-layered skin construct (BSC) accelerated the healing of venous ulcers in a randomized study population of 293 patients.

FIG. 2 shows analysis of patients in FIG. 1, restricted to those who had ulcers for more than a year, a difficult-to-heal ulcer population that responds to BSC.

FIG. 3 shows BSC was also able to accelerate healing of diabetic neuropathic foot ulcers.

FIG. 4 shows mRNA analysis of cytokines and growth factors after in vitro injury to the BSC. Some peptides, specifically IL-1α, IL-6, IL-8, TGF-α, and TGF-β1 are increased by 24 hours within the construct.

FIG. 5 shows the effect of increasing concentrations of antibodies to various growth factors and cytokines on the process of epiboly in a BSC model. Also tested were antibodies to vitronectin and its integrin receptor. The data represent the mean of quadruplicate experiments±SD.

FIG. 6 shows in the full-thickness mouse tail model that Smad3 knock-out mice display accelerated healing. The points represent the mean±SD (p<0.01).

FIGS. 7A-7B show dermal fibroblasts cultured from Smad3 knock-out mice show an increase in both thymidine incorporation (A) and actual cell numbers (B). The points and bars represent the mean±SD of quadruplicate determinations (p<0.02).

FIG. 8 shows the early effect of BSC stimulation on the resurfacing of tail wounds in C57BL/6 mice. There was a statistically significant effect by Day 4 in the group of mice treated with primed (stimulated) BSC. The bars represent the mean±SD. Pre-activated refers to primed BSC.

### DESCRIPTION OF THE PREFERRED EMBODIMENTS

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific compositions, or to particular engineered tissue constructs, as such may, of course, vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “an engineered tissue construct” includes mixtures of engineered tissue constructs; reference to “a tissue culture medium” includes mixtures of two or more such media, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not. For example, the phrase “optionally injured” means that the tissue construct may or may not be injured and that the description includes both injured and uninjured tissue constructs.

Provided is a composition comprising a primed engineered tissue construct, wherein the tissue construct is primed by contact with a tissue culture medium in vitro. Tissue engineering is the use of a combination of cells, engineering materials and methods, and suitable biochemical and physical/chemical factors to improve or replace biological functions. Thus, tissue engineering can be used to develop biological substitutes that restore the function of a damaged tissue or organ. As used herein, an “engineered tissue construct” is a composition produced for restoring the function of a damaged biologic tissue or organ. As used herein, a “primed engineered tissue construct” is an engineered tissue construct which, prior to being administered or applied to a subject, has undergone additional treatment and/or manipulation in vitro or ex vivo to make the tissue construct more effective than it would be without priming.

For example, a composition can comprise a primed engineered tissue construct comprising a living skin construct. In one aspect, the skin construct can comprise human allogeneic neonatal foreskin keratinocytes. In another aspect, the skin construct can comprise human allogeneic neonatal foreskin fibroblasts. In another aspect, the skin construct can comprise human allogeneic neonatal foreskin keratinocytes and human allogeneic neonatal foreskin fibroblasts. In yet another aspect, the skin construct can comprise adult cells. The living skin construct can optionally be in a bovine collagen gel.

An engineered tissue construct can be mono-layered, i.e., comprising a single layer of cells and matrix. As used herein, “matrix” refers to substances in tissues and organs that serve a structural role or modify cell behavior and interactions. Examples of substances that can comprise a matrix include, but are not limited to, collagen, fibronectin, and vitronectin. An example of a mono-layered skin construct is an epithelial sheet. In another aspect, an engineered tissue construct can be bi-layered, i.e., comprising two layers of cells and matrix, for example a cutaneous substitute. In another aspect, for example, an engineered living skin construct can be bi-layered and comprise human allogeneic neonatal foreskin keratinocytes and/or human allogeneic neonatal foreskin fibroblasts optionally in bovine collagen gel. In addition to neonatal cells, a living skin construct can comprise adult cells. As used herein, “BSC” means a bi-layered skin construct. Optionally, an engineered living skin construct can comprise endothelial cells, sweat glands, and hair follicles.

An engineered tissue construct, for example a living skin construct, can be primed after being in contact with a tissue culture medium for from about 4 hours to about 48 hours. Thus, a living skin construct can be primed by being in contact with a tissue culture medium for about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, or for any time period in between. In one aspect, a living skin construct is primed after being in contact with a tissue culture medium in 5% CO₂ at 37° C. for about 24 hours. As used herein, a living skin construct is “in contact” with a tissue culture medium when the construct touches the medium. Thus, a living skin construct can be in contact with a tissue culture medium when the skin construct is floated on the surface of the medium so that only one surface of the skin construct touches the medium. Alternatively, a living skin construct can be in contact with a tissue culture medium when the skin construct is immersed, either partially or totally, in the medium so that parts or all of both surfaces (top and bottom) of the skin construct touch the medium. In one aspect, a living skin construct can be in contact with a tissue culture medium and stored (incubated) in 5% CO₂ at a temperature of about 37° C. In another aspect, a living skin construct, for example Apligraf® (Organogenesis, Canton, Mass.), can be stored at room or ambient temperatures, for example, from about 20° C. to about 24° C., i.e., at 20° C., 21° C., 22° C., 23° C., and 24° C. and all temperatures in between. In another aspect, a living skin construct, for example Dermagraft® (Advanced BioHealing, LaJolla, Calif.) can be stored frozen (cryopreserved) at a temperature of about −70° C. and thawed for a few minutes before being applied to a subject. In still another aspect, a living skin construct, for example Orcel® (Ortec International, NY, NY) can be stored in liquid nitrogen at about −150° C.

A tissue culture medium can be any tissue culture medium known to a person of skill in the art and can optionally be serum-free. Thus, a tissue culture medium can comprise serum or it can be serum-free. Examples of tissue culture media include, but are not limited to, Dulbecco's Modified Eagle's Medium (DMEM), adaptive immunotherapy media (AIM-V), Roswell Park Memorial Institute media (RPMI), HyClone media, and a physiological solution. Examples of physiological solutions include, but are not limited to, saline, phosphate-buffered saline, Dulbecco's phosphate buffered saline, Lactated Ringer's solution, and other biocompatible fluids known to persons of skill in the art.

Also provided is a composition comprising a primed engineered tissue construct that can optionally be injured. For example, an engineered skin construct can be injured by being meshed, lacerated, perforated, fenestrated, or stimulated by light or laser beam. For example, a primed engineered skin construct can be meshed at a ratio from about 1.5 to 1 to about 3 to 1. As is known to a person of skill in the art, an engineered skin construct is injured in order to expand it to cover and treat a larger surface area and to allow for body fluids from the wound surface to escape. Exemplary wavelengths for a laser beam include wavelengths from about 600 nanometers to about 900 nanometers, which avoid excessive thermal damage to a construct.

Further, the disclosed compositions can be freeze-dried following priming (with or without being injured) for storage, transport, and later use. Freeze drying (lyophilization) involves the controlled removal of water from a construct, thereby preventing bacterial contamination and growth. A freeze-dried composition can be reconstituted for use according to methods well known in the art.

Further provided is a method of priming an engineered tissue construct, comprising contacting the tissue construct with a tissue culture medium, whereby contacting the tissue construct with the tissue culture medium primes the tissue construct. In one aspect, the tissue construct comprises a living skin construct. A living skin construct can comprise, for example, human allogeneic neonatal foreskin keratinocytes, human allogeneic neonatal foreskin fibroblasts, or both, and can optionally comprise adult cells. The cells can be allogeneic or autologous for priming. An engineered tissue construct can be mono-layered, i.e., comprising a single layer of cells and matrix. An example of a mono-layered skin construct is an epithelial sheet. In another aspect, an engineered tissue construct can be bi-layered, i.e., comprising two layers of cells and matrix, for example a cutaneous substitute. In another aspect, an engineered skin construct can be bi-layered and comprise human allogeneic neonatal foreskin keratinocytes and/or human allogeneic neonatal foreskin fibroblasts optionally in bovine collagen gel.

A tissue culture medium can be any tissue culture medium known to a person of skill in the art and can optionally be serum-free. Thus, a tissue culture medium can comprise serum or it can be serum-free. Examples of tissue culture media include, but are not limited to, Dulbecco's Modified Eagle's Medium (DMEM), adaptive immunotherapy media (AIM-V), Roswell Park Memorial Institute media (RPMI), HyClone media, and a physiological solution. An engineered tissue construct, for example a living skin construct, can be primed after being in contact with a tissue culture medium for from about 4 hours to about 48 hours. Thus, an engineered skin construct can be primed by being in contact with a tissue culture medium for about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, or for any time period in between. In one aspect, a living skin construct can, for example, be primed after being in contact with a tissue culture medium (incubated) for about 24 hours in 5% CO₂ at about 37° C.

In addition to being primed by contact with a tissue culture medium, the disclosed compositions can optionally be injured by one or more methods including, but not limited to, being meshed, lacerated, perforated, fenestrated, or stimulated by light or laser beam. Exemplary wavelengths for a laser beam include wavelengths from about 600 nanometers to about 900 nanometers, which avoid excessive thermal damage to a construct. An engineered tissue construct can be primed before, after, or simultaneously with one or more methods of injuring the construct. Meshing, lacerating, perforating, and fenestrating a skin construct, for example, can be carried out using various instruments and devices known to a person of skill. Meshing, for example, can be achieved by placing a construct or graft on an adapter which is run through a device that punches regularly spaced holes in the construct. In one aspect, meshing can be performed at a ratio of from about 1.5 to 1 to about 3 to 1. Examples of other instruments and devices that can be used to injure an engineered living skin construct include, but are not limited to, scalpels, scissors, and tissue punches. Following priming and optional injury, a disclosed composition can be freeze-dried, according to methods well known in the art, for storage, transport, and later use.

Further provided is a composition comprising a primed engineered tissue construct, prepared by a method comprising contacting a tissue construct with a tissue culture medium. In one aspect, a primed engineered tissue construct can be a living skin construct comprising, for example, human allogeneic neonatal foreskin keratinocytes, human allogeneic neonatal foreskin fibroblasts, or both, and can optionally comprise adult cells. The cells can be allogeneic or autologous for priming. An engineered tissue construct can be mono-layered, i.e., comprising a single layer of cells and matrix. An example of a mono-layered skin construct is an epithelial sheet. In another aspect, an engineered tissue construct can be bi-layered, i.e., comprising two layers of cells and matrix, for example a cutaneous substitute. In another aspect, for example, an engineered skin construct can be bi-layered and comprise human allogeneic neonatal foreskin keratinocytes and/or human allogeneic neonatal foreskin fibroblasts optionally in bovine collagen gel.

A tissue culture medium can be any tissue culture medium known to a person of skill in the art and can optionally be serum-free. Thus, a tissue culture medium can comprise serum or it can be serum-free. Examples of tissue culture media include, but are not limited to, Dulbecco's Modified Eagle's Medium (DMEM), adaptive immunotherapy media (AIM-V), Roswell Park Memorial Institute media (RPMI), HyClone media, and a physiological solution. An engineered tissue construct, for example a living skin construct, can be primed after being in contact with a tissue culture medium for from about 4 hours to about 48 hours. Thus, an engineered skin tissue construct can be primed by being in contact with a tissue culture medium for about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, or for any time period in between. In one aspect, a living skin construct can be primed after being in contact with a tissue culture medium (incubated) in 5% CO₂ at about 37° C. for about 24 hours.

In addition to being primed by contact with a tissue culture medium, the disclosed compositions can optionally be injured by one or more methods including, but not limited to, being meshed, lacerated, perforated, fenestrated, or stimulated by light or laser beam. Exemplary wavelengths for a laser beam include wavelengths from about 600 nanometers to about 900 nanometers, which avoid excessive thermal damage to a construct. An engineered tissue construct can be primed before, after, or simultaneously with one or more methods of injuring the construct. Meshing, lacerating, perforating, and fenestrating a skin construct, for example, can be carried out using various instruments known to a person of skill. Meshing, for example, can be achieved by placing a construct or graft on an adapter which is run through a device that punches regularly spaced holes in the construct. In one aspect, meshing can be performed at a ratio of from about 1.5 to 1 to about 3 to 1. Examples of other instruments and devices that can be used to injure an engineered living skin construct include, but are not limited to, scalpels, scissors, and tissue punches. Following priming and optional injury, a disclosed composition can be freeze-dried, according to methods well known in the art, for storage, transport, and later use.

The disclosed methods and compositions can be used, for example, in dermatologic surgery. Thus, provided is a method for healing a wound in a subject, comprising applying to the wound in the subject a therapeutically effective amount of a primed engineered tissue construct, whereby applying the primed tissue construct to the wound heals the wound in the subject. A living tissue construct can be applied to the surface of a wound with or without overlapping the edges of the wound. A tissue construct can cover the entire surface of the wound or optionally cover part of the surface of the wound. Dressings, film sprays, glue materials, sutures, and staples, for example, can be used to keep a construct in place on the surface of the wound.

As used herein, a “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included.

As used herein, “healing a wound” means treating a wound to restore integrity to the defective (wounded) organ or tissue, i.e., to rehabilitate or restore the wounded organ or tissue to a healthier condition. The treatment can result in partial or complete restoration of the integrity of the organ or tissue.

A wound can be an acute or chronic wound. As used herein, an “acute” wound is a wound that has no underlying healing defect and is expected to heal with basic wound care, as is known to a person of skill in the art. As used herein, a “chronic” wound is a wound that has an underlying cause or defect, for example, diabetes mellitus, venous stasis, diseases of the microcirculation, trauma, or pressure, and requires additional efforts aimed at treating the underlying cause or defect. A wound is considered a chronic wound when it does not heal after being treated by routine methods known in the art. An example of a wound is a skin ulcer.

In one aspect, a tissue construct comprises a living skin construct. A living skin construct can comprise, for example, human allogeneic neonatal foreskin keratinocytes, human allogeneic neonatal foreskin fibroblasts, or both, and can optionally comprise adult cells. An engineered tissue construct can be mono-layered, i.e., comprising a single layer of cells and matrix. An example of a mono-layered skin construct is an epithelial sheet. In another aspect, an engineered tissue construct can be bi-layered, i.e., comprising two layers of cells and matrix, for example a cutaneous substitute. In another aspect, for example, an engineered skin construct can be bi-layered and comprise human allogeneic neonatal foreskin keratinocytes and/or human allogeneic neonatal foreskin fibroblasts optionally in bovine collagen gel.

A tissue culture medium can be any tissue culture medium known to a person of skill in the art and can optionally be serum-free. Thus, a tissue culture medium can comprise serum or it can be serum-free. Examples of tissue culture media include, but are not limited to, Dulbecco's Modified Eagle's Medium (DMEM), adaptive immunotherapy media (AIM-V), Roswell Park Memorial Institute media (RPMI), HyClone media, and a physiological solution. An engineered tissue construct, for example an engineered skin construct, can be primed after being in contact with a tissue culture medium for from about 4 hours to about 48 hours. Thus, an engineered skin tissue construct can be primed by being in contact with a tissue culture medium for about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, or for any time period in between. In one aspect, the engineered skin construct can be primed after being in contact with a tissue culture medium (incubated) in 5% CO₂ at about 37° C. for about 24 hours.

In addition to being primed by contact with a tissue culture medium, the disclosed compositions can be injured by one or more methods including, but not limited to, being meshed, lacerated, perforated, fenestrated, or stimulated by light or laser beam. An engineered tissue construct can be primed before, after, or simultaneously with a method of injuring the construct. Meshing, lacerating, perforating, and fenestrating a skin construct, for example, can be carried out using various instruments known to a person of skill. Meshing, for example, can be achieved by placing a construct or graft on an adapter which is run through a device that punches regularly spaced holes in the construct. In one aspect, meshing can be performed at a ratio of from about 1.5 to 1 to about 3 to 1. Examples of other instruments and devices that can be used to injure an engineered living skin construct include, but are not limited to, scalpels, scissors, and tissue punches. Following priming and optional injury, a disclosed composition can be freeze-dried, according to methods well known in the art, for storage, transport, and later use.

The disclosed compositions can be provided in a kit for supplying surgical tissue graft components. In one aspect, the kit comprises a first compartment comprising a tissue construct and a second compartment comprising a predetermined quantity of a tissue culture medium. The predetermined quantity of tissue culture medium is sufficient to prime the tissue construct when the tissue construct is in contact with the tissue culture medium. The kit itself may comprise plastic, glass, metal, or any other material sufficient to seal the components of the kit. One skilled in the art will recognize the materials with which to make the kit.

In one exemplary aspect, the first compartment comprises a living skin construct which can comprise human allogeneic neonatal foreskin keratinocytes, human allogeneic neonatal foreskin fibroblasts, or both, and can optionally comprise adult cells, optionally in bovine gel.

In another aspect, the second compartment is fluidically sealed and can contain, for example, from about 15 milliliters to about 40 milliliters of tissue culture medium in a 100 millimeter dish. In yet another aspect, the contents of the first compartment and the contents of the second compartment are not in communication with each other and can optionally be part of a single container.

In another aspect, the first compartment of the disclosed kit can optionally comprise a mechanism for meshing, lacerating, perforating, or fenestrating a tissue construct. Examples of such mechanisms include, but are not limited to, scalpels, scissors, and tissue punches. The kit can optionally comprise a mechanism for stimulating a tissue construct with light or laser beam.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLES

### Clinical Studies of a Bi-Layered Skin Construct (BSC)

There is substantial experience with chronic wounds treated with a living bi-layered skin construct, Apligraf® (Organogenesis, Canton, Mass.), comprising neonatal foreskin keratinocytes and fibroblasts in a bovine collagen gel.¹⁷ Apligraf® is an example of a bioengineered bi-layered living skin construct and is referred to in this example as “BSC.” The construct was the first skin substitute approved by the FDA for use in chronic wounds.

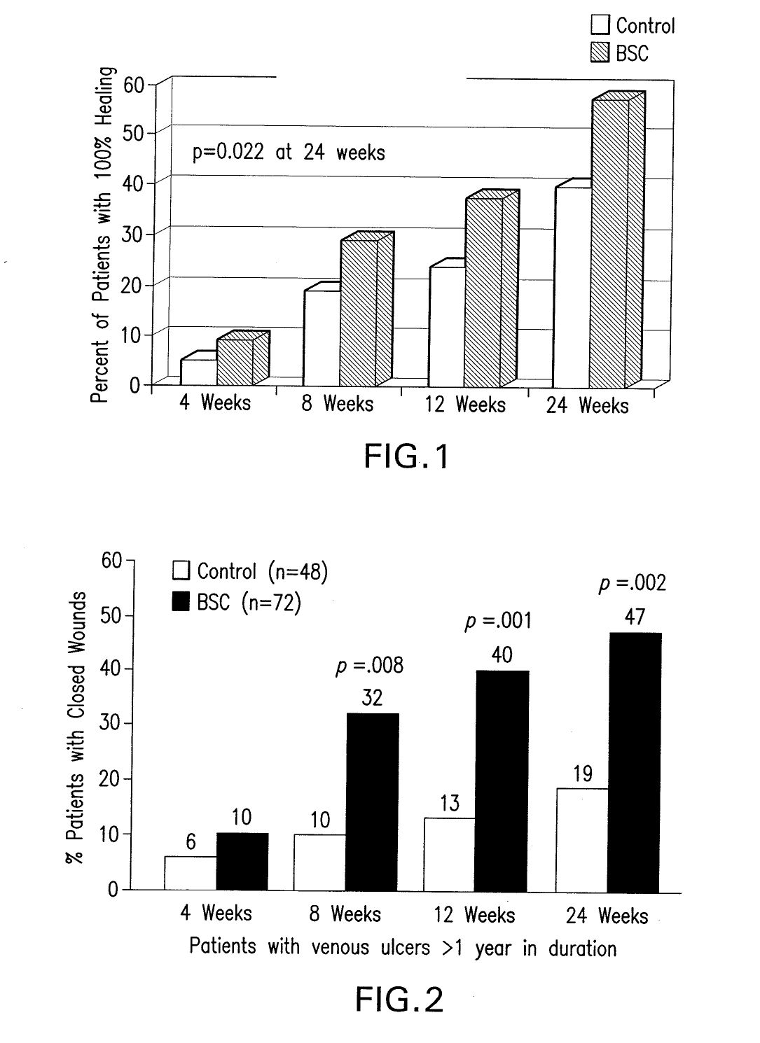

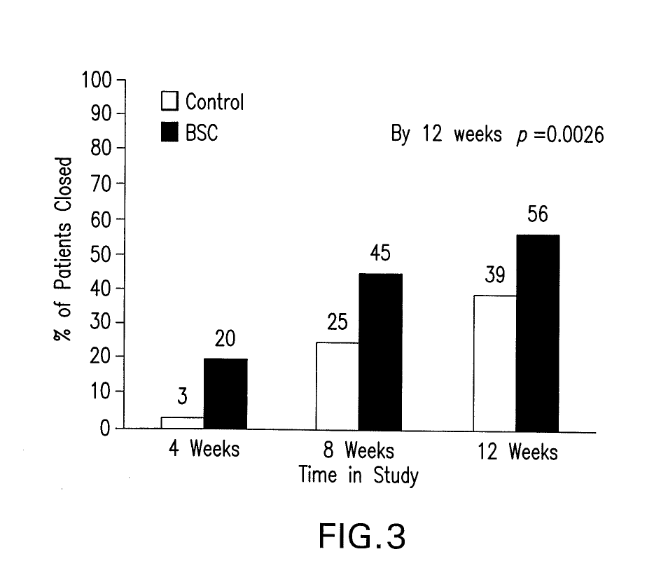

In two major randomized multicenter clinical trials, BSC was shown to be effective in accelerating closure of venous ulcers⁹ and diabetic neuropathic foot ulcers.¹⁰ The results of these studies are shown in FIGS. 1-3. BSC was able to stimulate the healing of venous ulcers.⁹ Further analysis showed that the statistical significance was largely driven by the subgroup of patients with hard to heal venous ulcers, present for more than a year.¹¹ However, as shown in FIG. 2, 50% of patients remained unhealed and unresponsive to this therapy. FIG. 3 shows that BSC also accelerated the healing of diabetic neuropathic foot ulcers.¹⁰ Presently, BSC is approved and being used for both indications.

### Lack of Persistence of BSC

In a prospective controlled study of patients with venous ulcers randomized to receive either the BSC or compression bandaging alone, it was determined by PCR of HLA subtypes that the construct only persists for up to eight weeks.²⁵ This was not entirely surprising because the donor cells of the construct are allogeneic.¹⁷ However, there had been the hypothesis that some of the cells might persist for a longer period of time. This evidence, together with other published information,²⁶ suggests that the BSC, as a prototype of allogeneic skin substitute, acts mainly as a stimulus for wound repair, possibly through the action of released cytokines.²

### Response of BSC to Injury In Vitro

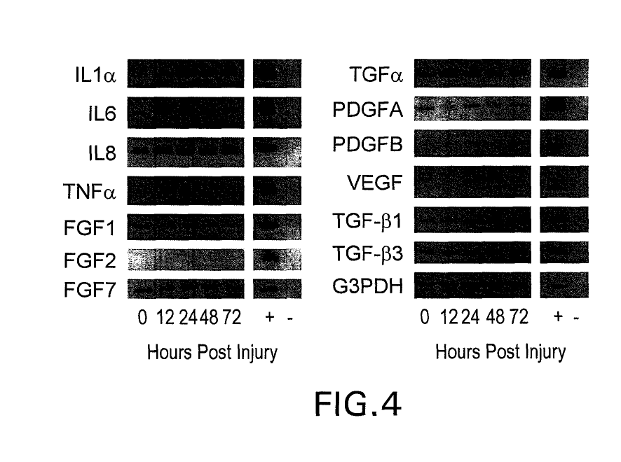

Following meshing or scalpel injury, the BSC is able to re-epithelialize itself in vitro.² Injury to the BSC by meshing and then incubation in DMEM in vitro for 24-48 hours stimulates the epidermal component to bridge the defect. Using ELISA, immunostaining, and PCR, a staged release of cytokines and growth factors was demonstrated that is quite similar to what occurs in the normal process of wound healing (FIG. 4).

Bioengineered skin constructs, such as BSC, can be stimulated due to their special properties, for example, by injury. Injury to the construct results in migration of the epidermal over its dermal component, a process known as epiboly.²⁷

### An Assay System for Keratinocyte Migration

In these experiments, 6-mm punch biopsies of the construct were placed in serum-free medium (AIM-V). The punches from the construct were simply floated into the medium, and they remained suspended in it without attaching to the tissue culture plastic. They were then removed at various times for histological and/or immunohistochemical analysis. The process of epiboly occurred within 24-72 hours. By 72 hours, in a time dependent manner, the epidermis showed completed migration over the construct and enveloped the entire dermis (full epiboly). Over several days, full stratification of the migrated epidermis occurred. This model represents a useful way for studying epidermal migration and may have distinct advantages over the often used “scratch” techniques using cell monolayers.²⁷

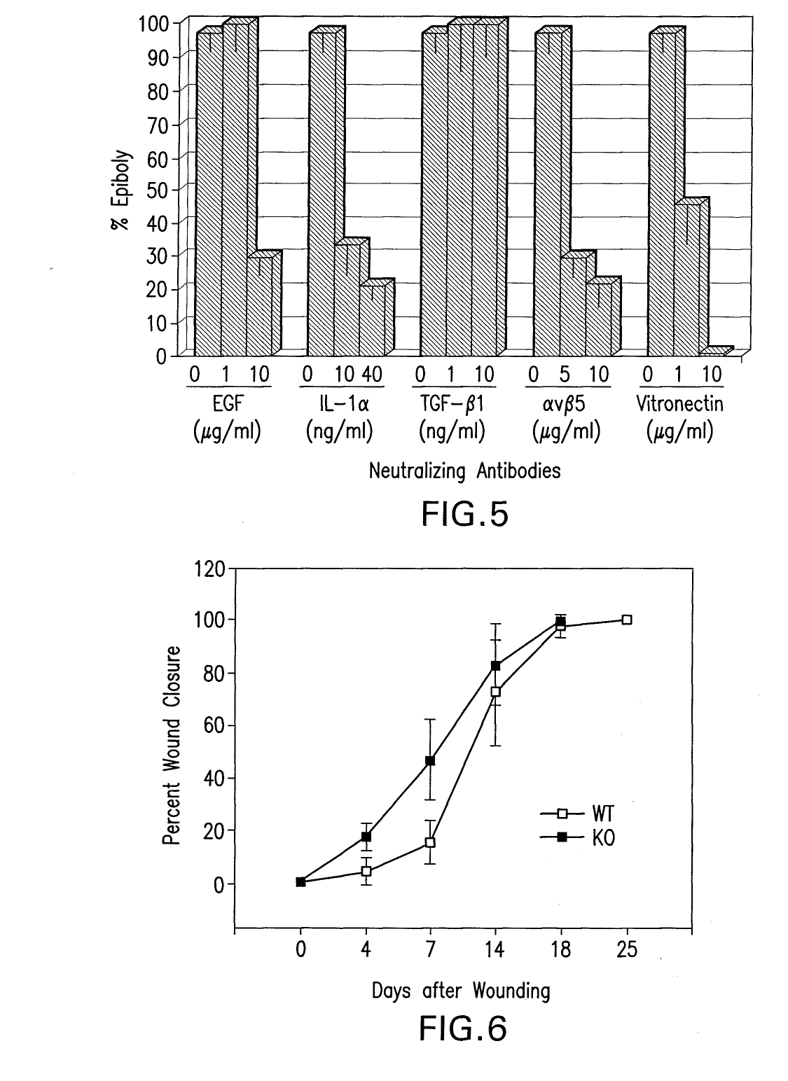

Full epiboly is partially inhibited by serum and is maximal in serum-free medium. It was found that the epiboly was downregulated by neutralizing antibodies to EGF or IL-1α, but unaffected by antibodies to TNF-α, PDGF-BB, or TGF-β1 (p<0.01; FIG. 5). At the end of 72 hours, using a 6-mm punch of the construct, the epidermis migrated and enveloped the underlying dermis. The migrating epithelium was also characterized by decreased keratinocyte proliferation (as per Ki67 immunostaining) and increased expression of vitronectin. FIG. 5 shows how epiboly was blocked by antibodies to vitronectin (epibolin) and the αvβ5 integrin receptor, which mediates vitronectin-driven keratinocyte locomotion.

### Keratin Expression During the Process of Epiboly

The process of wound healing is highly dependent on keratinocyte migration, and there is increasing evidence for an important role of specific cytokeratins. Using the epiboly model just described, the expression of certain keratins deemed important in proliferation/differentiation and migration was determined. Normal skin was used as an additional control. The process was already evident by 24 hours. Suprabasal expression of K10, a marker of proliferation, showed a clear sparing of the migrating tongue of epithelium. In contrast, dramatic expression of K17, now an established migration marker, was confined to the migrating edge of the epiboly. Little or no expression was seen in the original, more differentiated portion of the construct. Other keratins were also tested (K1, K6, K16). K1 and K10 were observed to be present in the normal skin and epiboly, and K1 was faintly present in the migrating edge, while K10 was notably absent. K16 and K6 were evident in normal skin and epiboly as well, and K6 was markedly present in the migrating edge. K17 presented itself only in epiboly in the migrating edge. Differential expression of these cytokeratins in epiboly suggests specificity to processes of migration and differentiation in re-epithelialization. The expression of these keratins, as well as c-myc and β-catenin expression and localization, may explain why priming of the construct may have its ultimate effect through these molecular markers.

### A Model of Wound Healing Using Full-Thickness Injury of Mouse Tail

Generally, injury to mouse skin heals quite readily, probably within 4 to 7 days. Indeed, it has been shown that Smad3 K-O mice have accelerated healing of full-thickness dorsal incisions, healing within two days.^{28, 29} A new model of wounding in the mouse was prepared which shows a more prolonged healing time.^{30, 31} The model consists of making full-thickness excisions of the mouse tails, down to fascia. After anesthesia, the mouse was placed in a chamber with the tail exposed. The dorsal aspect of the tail was marked for size (1.5 by 0.3 cm) and a #15 gauge scalpel was used to excise the tail skin and subcutaneous tissue down to fascia. Minor bleeding occurred, and the wound was dressed with a film polymer spray (Cavilon, 3M). Mice were sacrificed and their tails removed for analysis. Because the tail contains bone, there was a process of decalcification to properly prepare the specimen. Both histological and immunohistochemical determinations have been made. In collaboration with the laboratory of Dr. Seong-Jin Kim and other colleagues who had previously shown accelerated healing in Smad3 knock-out mice,²⁸ this model was validated using these knock-out animals. Acceleration of healing in Smad3 K-0 mice (p<0.01) is shown in FIG. 6.

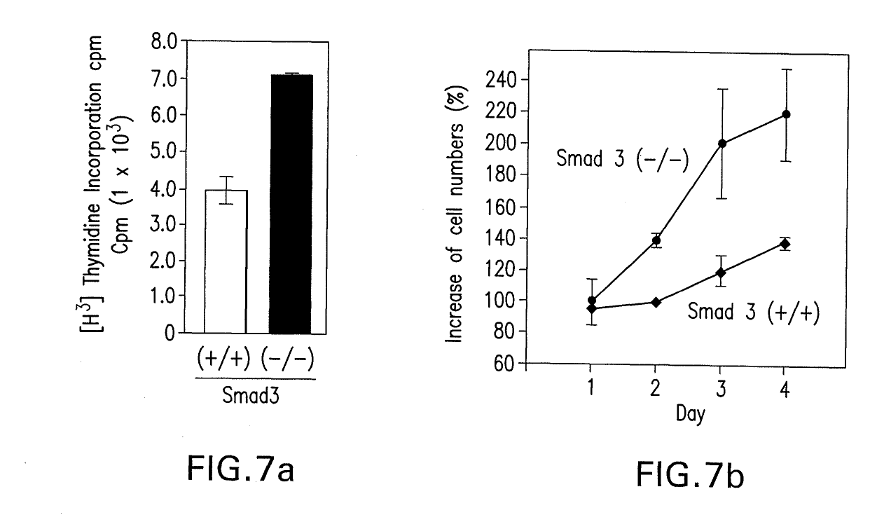

Additional data explain why Smad3 K-O mice heal faster. For example, the proliferation of Smad3 K-O fibroblasts was enhanced, as shown by proliferative assays and DNA synthesis (FIG. 7). Immunostaining with Ki67, a marker of cellular proliferation, shows greater intensity in the biopsies of wounds from Smad3 K-O mice.³⁰

### Use of Primed BSC in Mouse Tail Wounds

Nude mice were used to show that BSC can be properly applied to mouse tail wounds and stays in place and interacts with the wound. The construct is able to remain in place and then interact with the re-epithelializing wound. Coverage of the wound was done with a polymer film spray dressing (Cavilon, 3M), although a steel mesh can be used to cover the wounds.

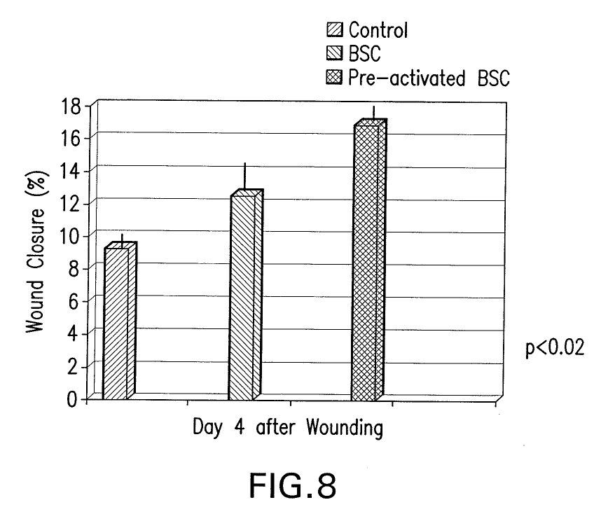

In other experiments, C57BL/6 mice were used to determine the effect of primed BSC. The BSC was stimulated in vitro 24 hours before the application to the tail wounds by making scalpel cuts1 cm in length and 1 cm apart in the BSC. The construct was then kept over its agar nutrient substrate, using incubating conditions of 37° C. and 5% CO₂. FIG. 8 shows the early effect of this BSC stimulation on the resurfacing of tail wounds in C57BL/6 mice. There was a statistically significant effect by Day 4 in the group of mice treated with primed BSC. These results were confirmed in db/db mice, and as far as Day 10 (p<0.04). These data suggest a biological effect of primed BSC.

### Stimulation of Epidermal Edges and Granulation Tissue in Chronic Human Venous Ulcers by the Use of Primed BSC

Four patients whose ulcers are more than one year in duration and which have been unresponsive to standard compression therapy and to repeated use of BSC itself have been studied. The healing rate, measuring the inward migration of the wound's edges was less than 0.04 cm/week (for the last four weeks), clearly below the threshold which is associated with impaired healing.^{1, 32, 33} Indeed, there had been an increase in wound size in two of the patients over the last four weeks prior to treatment.

The BSC was first meshed at a ratio of 1.5 to 1. The construct was then kept for 24 hours at 37° C., 5% CO₂ in DMEM (serum-free), using otherwise standard tissue conditions and in a way that was identical to what has been previously reported to stimulate epiboly.²⁷ The results observed were clinically very impressive, particularly so because this chronic wound has been so unresponsive to treatment, including the conventional use of BSC multiple times. Pain was also reduced dramatically. These patients indicate the feasibility of the approach from the clinical standpoint, including the technical aspects of priming the BSC.

This use of primed BSC is associated with very dramatic upregulation of K17 at the advancing edge of the chronic wound, as described above with the epiboly model. The expression of K17 is rather specifically upregulated in the advancing epidermal edge. The same finding was observed in the acute wound created in the ipsilateral thigh skin of the same patient with the leg ulcer. Further, there was no K17 expression in normal skin, before any type of injury.

### Conclusions

Injury of BSC in vitro leads to dramatic epiboly, with the keratinocyte layer virtually enveloping the dermal component. Cytokines and growth factors are involved in the epiboly process, and the epiboly can be blocked by antibodies to vitronectin (epibolin) and its integrin receptor (αvβ5). K17 is markedly expressed in the migrating edge of the epiboly model. These cases indicate that the priming “jump-starts” the healing process, in ways that were not possible before, when the very same patients received the unprimed construct.

After BSC is meshed and kept in tissue medium in vitro for 24 hours or longer, there is a staged response in terms of the production and release of inflammatory cytokines and growth factors, as demonstrated by Northern analysis and ELISA. This pattern very much mimics that reported to occur in the normal wound healing process.

BSC primed for 24 hours in vitro stimulates wound bed granulation tissue as well as the wound edge. New islands of skin develop in response to BSC, representing the recruitment of epidermal cells still present within the wound bed hair follicles or other appendageal structures.

### Genes Expressed by the Bi-Layered Skin Construct (BSC) After Priming

Based on a clinical trial to test the effect of primed BSC on the healing of venous ulcers, biopsies were obtained from the ulcer's edges and from acute wounds created in the same patient to prospectively determine the prognostic value of certain molecular markers, such as c-myc and β-catenin. The expression of these markers has been shown to correlate negatively with healing.³⁴⁻³⁶

Gene microarray experiments were performed to better understand the process of BSC priming. For the Affymetrix® analysis, three independent preparations for each sample type were used. The epiboly begins 24 hours after the construct is placed in DMEM without serum.¹⁵ Table 1 indicates 3 experimental groups tested using Affymetrix®. For the primed group, meshing was carried out at the standard ratio of 1.5 to 1. Standard culture conditions refer to incubation, i.e., contact between BSC and tissue culture medium, in 5% CO₂ and 37° C.

TABLE 1
Experimental Groups for the Affymetrix ® Analysis
of BSC in vitro

| Experimental Group | Description/Procedure |
|---|---|
| A (unmeshed/ | Basic control - no incubation and no meshing, and |
| no priming) | RNA extracted 15 minutes after removal from |
| | packaging |
| B (meshed/ | Clinical control - as used in the clinic, removed |
| no priming) | from packaging and RNA extracted 15 minutes after |
| | meshing |
| C (meshed/ | Full priming: removed from packaging, meshed, and |
| primed) | incubated in DMEM for 24 hours in standard culture |
| | conditions |

In this experiment, “full priming” refers to both meshing and subsequent incubation of BSC in DMEM for 24 hours (Group C). For Groups A and B, RNA was extracted 15 minutes after the construct was meshed or the package was opened because those are the recommendations for the product: to be used within 15 minutes after removal from the package.

Experiments were done in triplicate. Group A was used as the internal control for the gene analysis. Only genes that were at least 20 times up- or down-regulated are listed. There was no difference in gene expression between Groups A and B, as one might expect in such a short time after meshing and without priming. However, the primed construct (Group C) showed a number of genes that were highly up-regulated. Table 2 (below) is a list of genes that are up-regulated at least 20 times by Affymetrix® analysis.

TABLE 2
In Vitro Differentially Up-regulated Genes (at least
20-fold) after Full Priming of the Construct by Meshing
and 24 Hours of Tissue Culture Medium Incubation

| -Fold Increase in | |
|---|---|
| Gene Expression | Gene Identified |
| 136 | Interleukin-6 |
| 77 | Superoxide dismutase 2, mitochondrial |
| 54 | Serine protease inhibitor, Kazal type 1 |
| 52 | Enolase 2 (gamma, neuronal) |
| 39 | Heat shock 70 Kda protein 6 (HSP70B) |
| 37 | Tissue factor pathway inhibitor 2 |
| 29 | C-type lectin domain family 2, member B |
| 27 | Histone 1, H2bg |
| 26 | Basic helix-loop-helix domain containing, class B, 2 |
| 26 | Procollagen-lysine, 2-oxoglutarate 5-dioxygenase 2 |
| 26 | ATPase, H transporting, lysosomal 42 kDa, V1 |
| | Subunit C, isoform 1 |
| 26 | Family with sequence similarity 13, member A1 |
| 25 | Neurophilin 1 |
| 24 | Interleukin 11 |
| 23 | Interleukin 1 receptor-like 1 |
| 22 | DNA-damage-inducible transcript 4 |
| 21 | ATPase H transporting, lysosomal 42 kDa, V1 |
| | Subunit C, isoform 1 |
| 20 | Stanniocalcin 1 |

IL-6, for example, had previously been found to be up-regulated by Northern and ELISA.² In addition, more than another 80 genes were up-regulated between 10 and 20 times in the primed construct. Among them are vascular endothelial growth factor (14-fold), hypoxia-inducible protein 2 (15-fold), IL-1α (12-fold), calbindin (10-fold), cullin 4B (11-fold). Other genes previously found to be up-regulated after injury to the construct,² including IL-1, TGF-β1 and -β3, PDGF, were also increased, although their levels were less than 20-fold.

In another experiment conducted at the same time, BSC that were either meshed or left unmeshed before the 24 hour incubation in DMEM and standard culture conditions were compared. The fully primed (meshing plus incubation) constructs showed genes that were clearly differentially up-regulated. Notable examples are GM-CSF (27-fold), and MMP-12 (30-fold). GM-CSF up-regulation may make it possible for the primed BSC to better recruit macrophages and other cell types to the site of application. Interestingly, MMP-12 (macrophage metalloelastase) is also released by macrophages and is responsible for breaking down certain basement membrane structural proteins (entactin, laminin-1); it may therefore facilitate the process of epidermal migration. While the identification of certain specific genes is important, the value of priming the construct resides in jump-starting the epidermal migration of the construct.¹⁵ This jump-start in epidermal migration of the BSC makes it more effective. Thus, priming of the construct, which is associated with large increases in certain important genes, renders the BSC more effective.

Throughout this application, various publications are referenced. The disclosures of these publications in their entireties are hereby incorporated by reference into this application in order to more fully describe the state of the art to which this invention pertains.

### REFERENCES

1. Tallman P, Muscare E, Carson P, Eaglstein W H, Falanga V. Initial rate of healing predicts complete healing of venous ulcers. Arch Dermatol 1997; 133(10):1231-4.
2. Falanga V, Isaacs C, Paquette D, et al. Wounding of bioengineered skin: cellular and molecular aspects after injury. J Invest Dermatol 2002; 119(3):653-60.
3. Falanga V. Wound healing and its impairment in the diabetic foot. Lancet 2005; 366(9498): 1736-43.
4. Jones J, Nelson E. Skin grafting for venous leg ulcers. Cochrane Database Syst Rev 2007(2):CD001737.
5. Metcalfe A D, Ferguson M W. Tissue engineering of replacement skin: the crossroads of biomaterials, wound healing, embryonic development, stem cells and regeneration. J R Soc Interface 2006.
6. Mansbridge J. Skin substitutes to enhance wound healing. Expert Opin Investig Drugs 1998; 7(5):803-9.
7. Marston W A. Dermagraft, a bioengineered human dermal equivalent for the treatment of chronic nonhealing diabetic foot ulcer. Expert Rev Med Devices 2004; 1(1):21-31.
8. Badiavas E V, Paquette D, Carson P, Falanga V. Human chronic wounds treated with bioengineered skin: histologic evidence of host-graft interactions. J Am Acad Dermatol 2002; 46(4):524-30.
9. Falanga V, Margolis D, Alvarez O, et al. Rapid healing of venous ulcers and lack of clinical rejection with an allogeneic cultured human skin equivalent. Human Skin Equivalent Investigators Group. Arch Dermatol 1998; 134(3):293-300.
10. Veves A, Falanga V, Armstrong D G, Sabolinski M L. Graftskin, a human skin equivalent, is effective in the management of noninfected neuropathic diabetic foot ulcers: a prospective randomized multicenter clinical trial. Diabetes Care 2001; 24(2):290-5.
11. Falanga V, Sabolinski M. A bilayered living skin construct (APLIGRAF) accelerates complete closure of hard-to-heal venous ulcers. Wound Repair Regen 1999; 7(4):201-7.
12. Falanga V. Classifications for wound bed preparation and stimulation of chronic wounds. Wound Repair Regen 2000; 8(5):347-52.
13. Gallico G G, 3rd, O'Connor N E, Compton C C, Kehinde O, Green H. Permanent coverage of large burn wounds with autologous cultured human epithelium. N Engl J Med 1984; 311(7):448-51.
14. Phillips T J, Gilchrest B A. Clinical applications of cultured epithelium. Epithelial Cell Biol 1992; 1(1):39-46.
15. Phillips T J, Kehinde O, Green H, Gilchrest B A. Treatment of skin ulcers with cultured epidermal allografts. J Am Acad Dermatol 1989; 21(2 Pt 1):191-9.
16. Vanscheidt W, Ukat A, Horak V, et al. Treatment of recalcitrant venous leg ulcers with autologous keratinocytes in fibrin sealant: A multinational randomized controlled clinical trial. Wound Repair Regen 2007; 15(3):308-15.
17. Sabolinski M L, Alvarez O, Auletta M, Mulder G, Parenteau N L. Cultured skin as a ‘smart material’ for healing wounds: experience in venous ulcers. Biomaterials 1996; 17(3):311-20.
18. Hansbrough J. Dermagraft-TC for partial-thickness burns: a clinical evaluation. J Burn Care Rehabil 1997; 18(1 Pt 2):S25-8.
19. Hansbrough J F, Franco ES. Skin replacements. Clin Plast Surg 1998; 25(3):407-23.
20. Boyce S T. Design principles for composition and performance of cultured skin substitutes. Burns 2001; 27(5):523-33.
21. Nasseri B A, Vacanti J P. Tissue engineering in the 21st century. Surg Technol Int 2002; 10:25-37.
22. Phillips T J. New skin for old: developments in biological skin substitutes. Arch Dermatol 1998; 134(3):344-9.
23. Gentzkow G D, Iwasaki S D, Hershon K S, et al. Use of dermagraft, a cultured human dermis, to treat diabetic foot ulcers. Diabetes Care 1996; 19(4):350-4.
24. Naughton G, Mansbridge J, Gentzkow G. A metabolically active human dermal replacement for the treatment of diabetic foot ulcers. Artif Organs 1997; 21(11):1203-10.
25. Phillips T J, Manzoor J, Rojas A, et al. The longevity of a bilayered skin substitute after application to venous ulcers. Arch Dermatol 2002; 138(8):1079-81.
26. Saap L J, Donohue K, Falanga V. Clinical classification of bioengineered skin use and its correlation with healing of diabetic and venous ulcers. Dermatol Surg 2004; 30(8): 1095-100.
27. Falanga V, Butmarc J, Cha J, Yufit T, Carson P. Migration of the epidermal over the dermal component (epiboly) in a bilayered bioengineered skin construct. Tissue Eng 2007; 13(1):21-8.
28. Ashcroft G S, Yang X, Glick A B, et al. Mice lacking Smad3 show accelerated wound healing and an impaired local inflammatory response. Nat Cell Biol 1999; 1(5):260-6.
29. Kloeters O, Jia S X, Roy N, Schultz G S, Leinfellner G, Mustoe T A. Alteration of Smad3 signaling in ischemic rabbit dermal ulcer wounds. Wound Repair Regen 2007; 15(3):341-9.
30. Falanga V, Schrayer D, Cha J, et al. Full-thickness wounding of the mouse tail as a model for delayed wound healing: accelerated wound closure in Smad3 knock-out mice. Wound Repair Regen 2004; 12(3):320-6.
31. Falanga V, Iwamoto S, Chartier M, et al. Autologous Bone Marrow-Derived Cultured Mesenchymal Stem Cells Delivered in a Fibrin Spray Accelerate Healing in Murine and Human Cutaneous Wounds. Tissue Eng 2007.
32. Falanga V, Saap L J, Ozonoff A. Wound bed score and its correlation with healing of chronic wounds. Dermatol Ther 2006; 19(6):383-90.
33. Margolis D J, Gross E A, Wood C R, Lazarus G S. Planimetric rate of healing in venous ulcers of the leg treated with pressure bandage and hydrocolloid dressing. J Am Acad Dermatol 1993; 28(3):418-21.
34. Brem H, Stojadinovic O, Diegelmann R F, et al. Molecular markers in patients with chronic wounds to guide surgical debridement. Mol Med 2007; 13(1-2):30-9.
35. Brem H, Tomic-Canic M. Cellular and molecular basis of wound healing in diabetes. Clin Invest 2007; 117(5):1219-22.
36. Stojadinovic O, Brem H, Vouthounis C, et al. Molecular pathogenesis of chronic wounds: the role of beta-catenin and c-myc in the inhibition of epithelialization and wound healing. Am J Pathol 2005; 167(1):59-69.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A composition comprising a primed engineered tissue construct, wherein the tissue construct is primed by contact with a tissue culture medium in vitro.

2. The composition of claim 1, wherein the tissue construct comprises a living skin construct.

3. The composition of claim 2, wherein the skin construct comprises human allogeneic neonatal foreskin keratinocytes, human allogeneic neonatal foreskin fibroblasts, or both.

4. The composition of claim 3, further comprising adult skin cells.

5. The composition of claim 1, wherein the tissue construct is in contact with the tissue culture medium for from about 4 hours to about 48 hours.

6. The composition of claim 5, wherein the tissue construct is in contact with the tissue culture medium for about 24 hours.

7. The composition of claim 1, wherein the tissue culture medium is serum-free.

8. The composition of claim 7, wherein the serum-free medium is Dulbecco's Modified Eagle's Medium (DMEM), adaptive immunotherapy media (AIM-V), Roswell Park Memorial Institute media (RPMI), or HyClone media.

9. The composition of claim 1, wherein the primed tissue construct is meshed, lacerated, perforated, fenestrated, or stimulated by light or laser beam.

10. The composition of claim 9, wherein meshing is at a ratio of from about 1.5 to 1 to about 3 to 1.

11. The composition of claim 1, wherein the tissue construct is freeze-dried following priming.

12. A method of priming an engineered tissue construct, comprising contacting the tissue construct with a tissue culture medium, whereby contacting the tissue construct with the tissue culture medium primes the tissue construct.

13. The method of claim 12, wherein the tissue construct comprises a living skin construct.

14. The method of claim 13, wherein the skin construct comprises human allogeneic neonatal foreskin keratinocytes, human allogeneic neonatal foreskin fibroblasts, or both.

15. The method of claim 14, wherein the skin construct further comprises adult skin cells.

16. The method of claim 12, wherein the primed tissue construct is meshed, lacerated, perforated, fenestrated, or stimulated by light or laser beam.

17. The method of claim 16, wherein meshing is at a ratio of from about 1.5 to 1 to about 3 to 1.

18. The method of claim 12, wherein the tissue culture medium is serum-free.

19. The method of claim 18, wherein the serum-free medium is Dulbecco's Modified Eagle's Medium (DMEM), adaptive immunotherapy media (AIM-V), Roswell Park Memorial Institute media (RPMI), or HyClone media.

20. The method of claim 12, wherein the tissue construct is in contact with the tissue culture medium for from about 4 hours to about 48 hours.

21. The method of claim 20, wherein the tissue construct is in contact with the tissue culture medium for about 24 hours.

22. The method of claim 12, further comprising freeze-drying the tissue construct following priming.

23. A composition comprising a primed engineered tissue construct, prepared by a method comprising contacting a tissue construct with a tissue culture medium.

24. The composition of claim 23, wherein the tissue construct comprises a living skin construct.

25. The composition of claim 24, wherein the skin construct comprises human allogeneic neonatal foreskin keratinocytes, human allogeneic neonatal foreskin fibroblasts, or both.

26. The composition of claim 25, further comprising adult skin cells.

27. The composition of claim 23, wherein the primed tissue construct is meshed, lacerated, perforated, fenestrated, or stimulated by light or laser beam.

28. The composition of claim 27, wherein meshing is at a ratio of from about 1.5 to 1 to about 3 to 1.

29. The composition of claim 23, wherein the tissue culture medium is serum-free.

30. The composition of claim 29, wherein the serum-free medium is Dulbecco's Modified Eagle's Medium (DMEM), adaptive immunotherapy media (AIM-V), Roswell Park Memorial Institute media (RPMI), or HyClone media.

31. The composition of claim 23, wherein the tissue construct is in contact with the tissue culture medium for from about 4 hours to about 48 hours.

32. The composition of claim 31, wherein the tissue construct is in contact with the tissue culture medium for about 24 hours.

33. The composition of claim 23, wherein the tissue construct is freeze-dried following priming.

34. A method for healing a wound in a subject, comprising applying to the wound in the subject a therapeutically effective amount of a primed engineered tissue construct, whereby applying the tissue construct to the wound heals the wound in the subject.

35. The method of claim 34, wherein the tissue construct comprises a living skin construct.

36. The method of claim 35, wherein the skin construct comprises human allogeneic neonatal foreskin keratinocytes, human allogeneic neonatal foreskin fibroblasts, or both.

37. The method of claim 36, wherein the skin construct further comprises adult skin cells.

38. The method of claim 34, wherein the wound is a skin ulcer.

39. The method of claim 38, wherein the skin ulcer is an acute skin ulcer.

40. The method of claim 38, wherein the skin ulcer is a chronic skin ulcer.

41. A kit for supplying surgical tissue graft components, the kit comprising: a first compartment comprising a tissue construct; and a second compartment comprising a predetermined quantity of a tissue culture medium, wherein the predetermined quantity is sufficient to prime the tissue construct when the tissue construct is in contact with the tissue culture medium.

42. The kit of claim 41, wherein the tissue construct comprises a living skin construct.

43. The kit of claim 42, wherein the skin construct comprises human allogeneic neonatal foreskin keratinocytes, human allogeneic neonatal foreskin fibroblasts, or both.

44. The kit of claim 43, wherein the skin construct further comprises adult skin cells.

45. The kit of claim 41, wherein the second compartment is fluidically sealed.

46. The kit of claim 41, wherein the contents of the first and the second compartments are not in communication with each other.

47. The kit of claim 41, wherein the tissue construct is meshed, lacerated, perforated, fenestrated, or stimulated by light or laser beam.

48. The kit of claim 41, wherein the compartments are part of a single container.

49. The kit of claim 41, wherein the first compartment further comprises a mechanism for meshing, lacerating, perforating, or fenestrating the tissue construct.

50. The kit of claim 41, wherein the first compartment comprises a mechanism for stimulating the tissue construct with light or laser beam.
